(19) United States
(12) Patent Application Publication
(10) Pub. No.: US 2010/0220285 A1
(43) Pub. Date: Sep. 2, 2010

(54) FITTING OF SPECTACLES

(76) Inventor: Adam Simmonds, London (GB)

Correspondence Address: KLEIN, O''NEILL & SINGH, LLP, 18200 VON KARMAN AVENUE, SUITE 725, IRVINE, CA, 92612, US

(21) Appl. No.: 12/667,919

(22) PCT Filed: Jul. 11, 2008
(86) PCT No.: PCT/GB2008/002380

§ 371 Date: May 14, 2010

(30) Foreign Application Priority Data
Jul. 11, 2007 (GB) 0713461.2

(51) Int. Cl. A61B 3/11

(52) U.S. Cl. 351/204; 351/246

(57) ABSTRACT

A handheld device for aligning a lens with the eye of a patient. The device includes a capture apparatus for capturing and storing an image of a patient wearing spectacles, and processor for determining on the image the center of a pupil of the patient, and indicating on a display the position of the lens over the eye of the patient wherein the optical center of the lens is aligned with the pupil of the patient.

### CROSS-REFERENCE TO RELATED APPLICATION(S)

This is a national phase application of PCT No. PCT/GB2008/002380, filed Jul. 11, 2008, which claims priority to GB application No. 0713461.2, filed Jul. 11, 2007, the contents of each of which are expressly incorporated herein by reference as if set forth in full.

### BACKGROUND

The invention relates to the fitting of spectacles. In particular, the invention relates to the correct alignment of spectacle lenses with the pupils of a spectacle wearer's eyes.

When fitting spectacles it is important to ensure that the optical centers of the fitted lenses are correctly positioned relative to a patient's pupils. Ideally, the optical center of a lens should be positioned over the center of the patient's pupil. This is particularly important when the lenses are varifocal lenses. The position of the optical centers of the lenses also depends on the function of the spectacles being dispensed (e.g. near or far distance vision). For example, the optical centers of reading spectacle lenses will be closer to the bridge of the spectacles than those prescribed for long sightedness. If the optical centers of the lenses are not accurately aligned, the effectiveness of the lenses is reduced.

The current spectacle dispensing process involves the marking of the optimum position of the optical centers of the lenses by hand. Typically, once the patient has selected a pair of frames an optician will use a permanent marker to indicate the position of the patient's pupils on the blanks housed in the frames while the patient is wearing the spectacles. The optician usually judges the position of the patient's pupils by eye, or they may use a measuring device. The measuring device may be a ruler or a more specialised device such as that disclosed in U.S. Pat. No. 4,131,338. Once the optician has indicated where he considers the optimum position of the optical centers of the lenses to be, the spectacles, including the marked blanks, are then sent to a lens manufacturer for production and fitting of the lenses.

It is clear that the above method of aligning the optical centers of lenses is far from perfect. The accuracy of alignment can be affected by a number of parameters, for example, movement of the patient's eyes when the optician is marking or measuring the pupils' position, and not least the skill of the optician.

A number of devices have been developed in an attempt to improve the accuracy of the alignment of optical centers over a patient's pupils. For example, United Kingdom Patent No. 885,429 describes a device for measuring the distance of a spectacle wearer's pupils from each other and the bridge of their nose. More recent devices, known as “pupilometers”, have been commercialised by companies such as Essilor Limited, NIDEK Co., Ltd. and Hoya. Pupilometers measure a patient's pupilary distance. The pupilary distance is the distance from the pupil center of one eye of the patient to the pupil center of the other eye of the patient. However, the pupilometers mentioned above are unable to measure the pupilary distance accurately enough to correctly position the optical centers over a patient's pupils. Furthermore, pupilometers do not measure the pupilary distance and position of the pupil centers in relation to the optical centers of lenses or the dimensions of spectacle frames.

### SUMMARY

The present invention resides, among others, in a device and method intended for acquiring dimensional information for patient pupil centers relative to a chosen pair of spectacle frames.

Against this background, the present invention resides in a device for aligning a lens with the eye of a patient, the device comprising means for capturing and storing an image of a patient wearing spectacles; and processing means for determining on the image the center of a pupil of the patient, and indicating on a display the position of the lens over the eye of the patient wherein the optical center of the lens is aligned with the pupil of the patient.

The present invention is based upon digital image capture and image recognition technology. Instead of using a ruler, for example, the optician captures an image of the patient wearing the spectacles and the device automatically recognises the pupil centers and calculates the distance to the frame edge. The information is output for communication to the frame glaziers or manufacturer, for example on a colour printout.

The device advantageously increases accuracy by utilising digital technology to replace the imprecise process of manual measuring during the dispensing of spectacle frames. This results in improved vision quality for the patient and therefore fewer returned spectacles to the optician. The invention provides a simple “point and shoot” data collection process, which means that a user of the invention may be relatively unskilled. For example, the method of the invention is not reliant on the availability of a qualified optician.

Preferably, the processing means comprises an edge enhancement algorithm for highlighting edges in the image and/or a circle recognition algorithm for detecting circular shapes in the image and/or a dark recognition algorithm for detecting dark areas in the image and/or an algorithm for detecting the center of the pupil of the patient in the image.

Alternatively, the processing means may comprise a red-eye light source, wherein the red-eye light source may be a standard camera flash. The device may comprise comparison means for comparing a standard image and a “red-eye” image.

In a preferred embodiment, the device comprises distance measurement means for calculating the distance from the device to the patient.

The distance measurement means may comprise a double optical assembly which are positioned a known distance apart, and optical processing means for calculating the distance from the device to the patient using stereoscopic imaging. Alternatively, the distance measurement means comprises a single optical assembly wherein the assembly is motor-driven; and a focus detection means which is arranged to drive the assembly to achieve a sharp image; wherein the single optical assembly is calibrated with the focus detection means so that the distance from the device to the patient is calculated. Alternatively, the distance measurement means comprises an ultrasonic transmitter for transmitting an ultrasonic signal and an ultrasonic receiver for receiving the ultra sonic signal, and ultrasonic processing means for calculating the distance from the device to the patient. Alternatively, the distance measurement means comprises aiming guides superimposed on the display.

Preferably, the device comprises means for indicating, on the image, edges of a spectacle frame for the lens.

Preferably, the device comprises convergence averting means. The convergence control unit may comprise laser speckle generating means. Alternatively, the convergence control unit may comprise a first light source and a second light source. Alternatively, the convergence control unit may comprise a reflective surface. Alternatively, the convergence control unit may comprise processing means for correcting convergence.

Preferably, the device comprises an orientation detector for detecting the orientation of the device or scaling the image. The orientation detector may be an electromagnetic tilt sensor or an accelerometer.

Preferably, the device comprises cursor keys and a select key for moving a cursor on the display of the device for indicating edges of the spectacle frames.

Preferably, the device is arranged to calculate dimensions on the image. The dimensions may be one or more of the frame datum, vertical datum, PD, H1, H2, H3 and MDBL. Preferably, the device is arranged to calculate pantoscopic tilt. Preferably, the device is arranged to superimpose a circle, the image representing a lens, over the eye of the patient on the image.

According to a further aspect the invention resides in a system comprising a device as described above; a docking station engageable the device arranged to communicate with and provide power to the device; and a print output device.

Preferably, the print output device is arranged to print an output file corresponding to the image on the display.

According to a further aspect the invention resides in a method for aligning a lens with the eye of a patient, the method comprising capturing and storing an image of a patient wearing spectacles on a device; processing the image to determine the center of the pupil of the patient; and indicating the correct position of the optical center of the lens over the pupil of the patient on a display of the device.

In a preferred embodiment, the method may comprise inducing an infinity gaze in the patient. In a further preferred embodiment, the method comprises a user of the device altering the position of the lens in the image. In a further preferred embodiment, the method comprises measuring the distance from the device to the patient. The method may comprise scaling the image using the distance from the device to the patient.

The method may comprise indicating edges of the spectacles on the image. The method may also comprise selecting a lens blank from a selection of lens blanks illustrated on the display, and the blanks may be downloaded from the Internet.

The method may comprise calculating dimensions on the image, and the dimensions may be one or more of the frame datum, vertical datum, PD, H1, H2, H3 and MDBL.

The method may also comprise communicating the type of lens and its position relative to the spectacles to a manufacturer.

Advantageously, lenses are manufactured to the patient's prescription and may be supplied back to the optician in circular format for glazing (the process of cutting the lenses to the shape of the spectacle frames) or already fitted in the frames. It is important for the optician to order the lens diameter most appropriate for the chosen spectacle frames in order to minimise lens edge thickness. Current practice is for the optician to estimate the lens diameter required by comparing the frames to printed templates supplied by the lens manufacturers. However, the method of the present invention may also comprise calculating lens thickness.

The invention may communicate with a PC for ease and convenience of use.

### SUMMARY OF THE DRAWINGS

In order that the invention may be more readily understood, reference will now be made, by way of example, to the accompanying drawings in which:

FIG. 1 is a view of a system for measuring and recording interpupillar distance according to the invention;

FIG. 2a is a top view of a handheld device according to a first embodiment of the invention;

FIG. 2b is a front view of the handheld device shown in FIG. 2a;

FIG. 3a is a perspective view of a handheld device according to a second embodiment of the invention;

FIG. 3b is an exploded view of the handheld device shown in FIG. 3a according to a second embodiment of the invention;

FIG. 4a is a flow diagram illustrating a method of image capture according to the invention;

FIG. 4b is a flow diagram illustrating a method of determining the position of pupils according to the invention;

FIG. 5a is a diagram illustrating the measurement points on a human face for establishing the correct position of the optical centers of spectacle lenses;

FIG. 5b is a flow chart illustrating a method of determining the pupil centers of a patient relative to their spectacles; and

FIGS. 6a and 6b illustrate a printout obtained from the invention.

### DETAILED DESCRIPTION

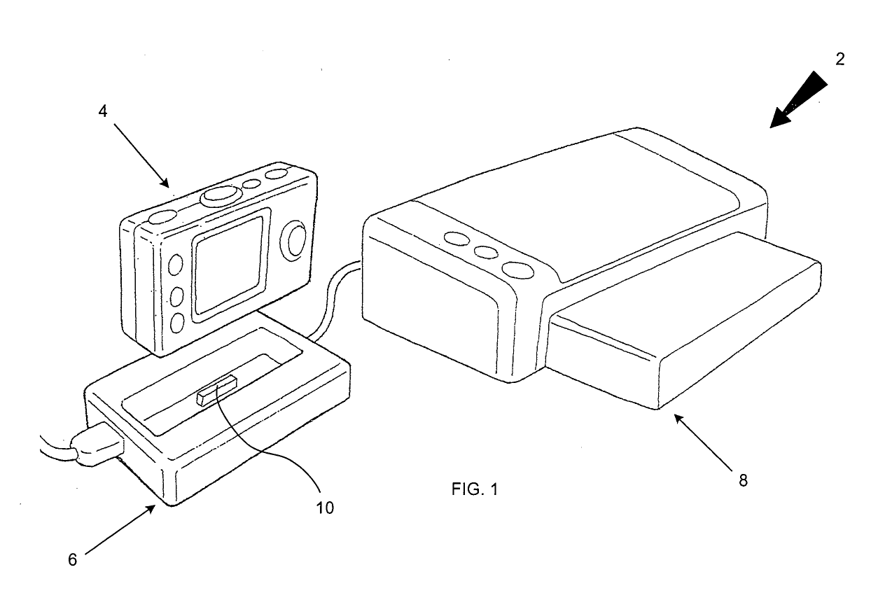

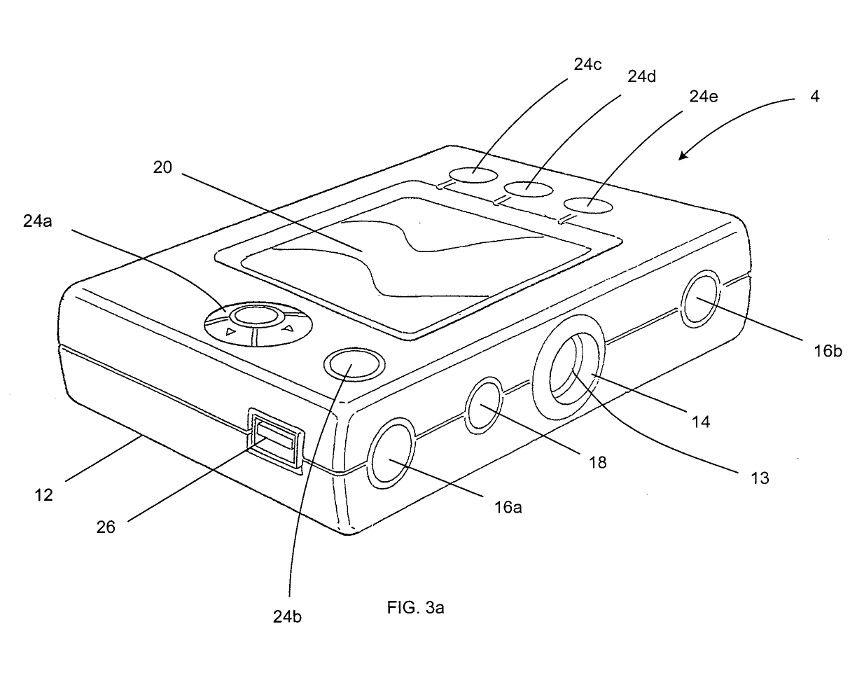

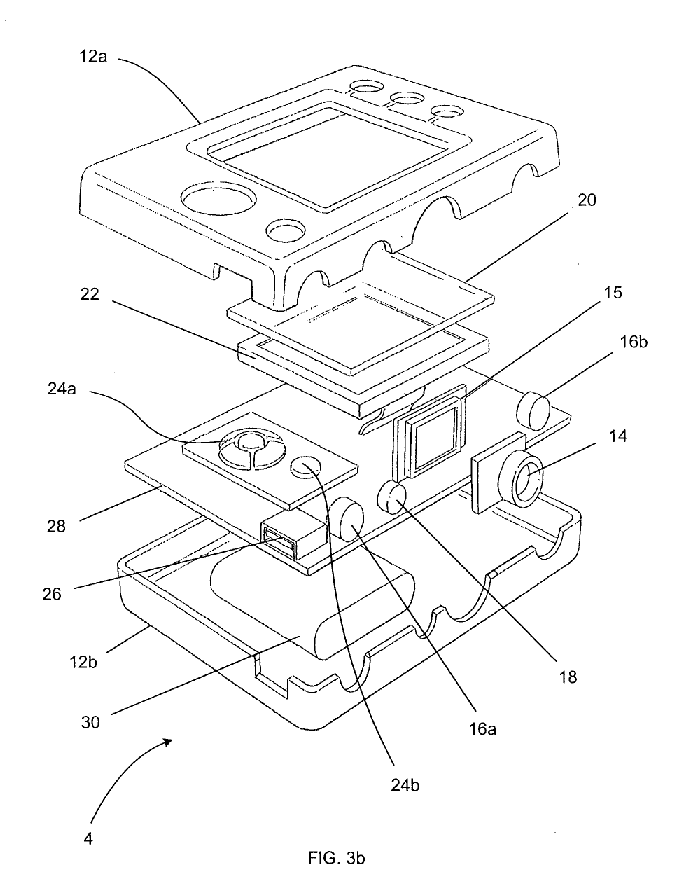

A system 2 for measuring and recording the position of a patient's pupils relative to the lenses of spectacles worn by a patient is shown in FIG. 1. The system 2 comprises a handheld device 4; a docking station 6 for the handheld device 4, which incorporates an interface 10 for providing power from the docking station 6 to recharge a battery 30 (shown in FIG. 3) of the handheld device 4 and facilitates data communication between the handheld device 4 and docking station 6; and a print output device 8.

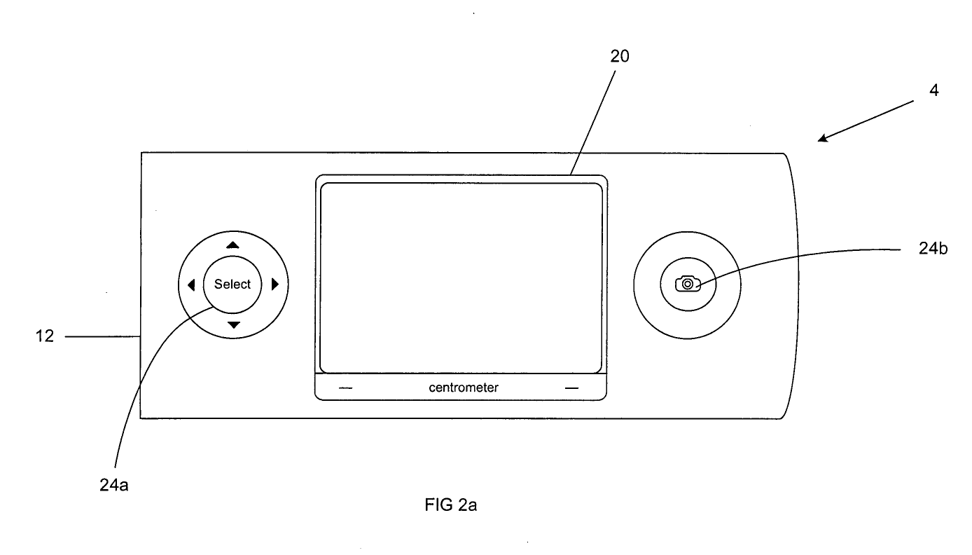

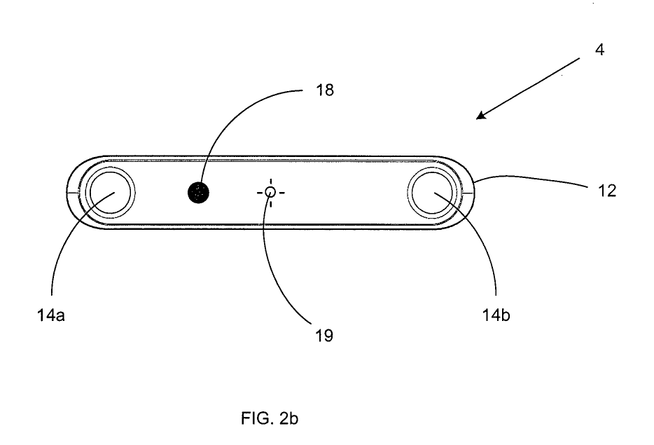

The handheld device 4 according to a first embodiment of the invention is described in more detail with reference to FIGS. 2a and 2b. Mounted in the casing 12 is a first optical lens assembly 14a and a second optical lens assembly 14b, also known as a “double optical assembly”, each assembly contains a lens 13 (shown in FIG. 3a) which may have an automatic or fixed focus assembly. Mounted behind each assembly 14a, 14b and inside the casing 12 is an CMOS/CCD image sensor module 15 (shown in FIG. 3b); a display window 20, behind which and inside the casing 12 is an LCD display module 22 (shown in FIG. 3b) which can be viewed through the display window 20; curser keys 24a a select key 24e and an image capture key 24b, for operating and controlling the handheld device 4; and a convergence control unit 19.

A second embodiment of the invention is now described with reference to FIGS. 3a and 3b. Features which are contained in both the first and second embodiments are now described. Within the casing 12 is housed an electronic PCB assembly 28 which contains a microprocessor (not shown) on which runs software, a memory (not shown) and the battery 30. Electro-mechanical tilt sensors (not shown) may also be housed within the casing 12, as well as data storage devices (not shown) which may be removable.

In the second embodiment of the invention the casing 12 is split into an upper casing 12a and a lower casing 12b, and rather than having two optical lens assemblies as in the first embodiment, the second embodiment has a single lens assembly 14. The second embodiment also comprises a red-eye light source 18; an ultrasonic transmitter 16a and an ultrasonic receiver 16b and function keys 24c, 24d, 24e; a data communications port 26; and a connector (not visible in FIGS. 3a and 3b) which is co-operable with the interface 10.

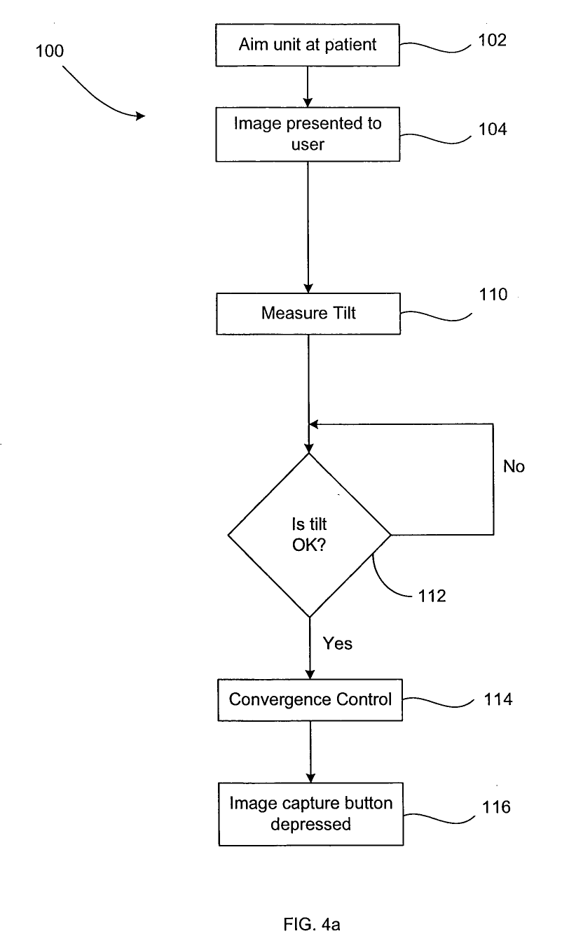

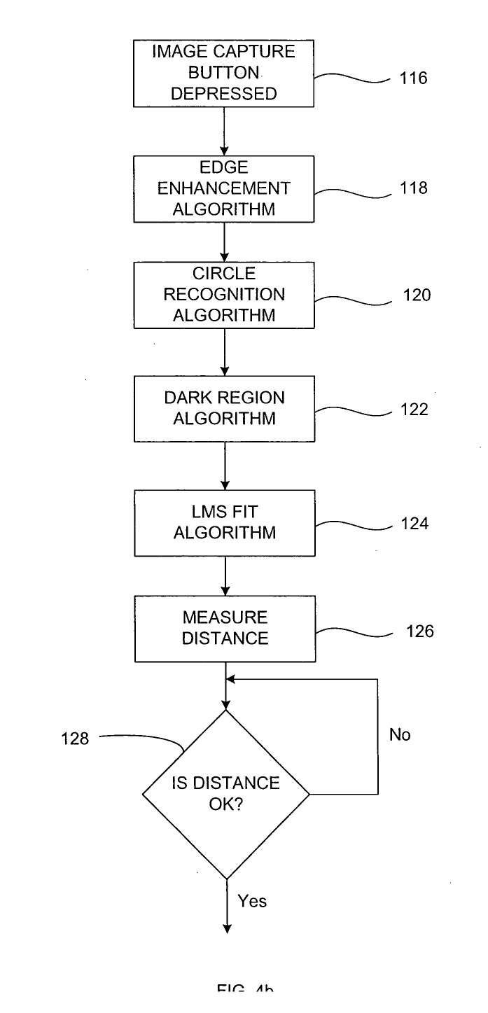

A method 100 according to the first embodiment of the present invention for capturing an image of the patient wearing a pair of spectacles, the image being suitable for analysis, is now described with reference to FIG. 4.

After a patient has selected a pair of spectacles in a dispensing opticians, the optician, who is referred to herein as the “user” of the handheld device 4, aims at step 102 the first and second optical lens assemblies 14a, 14b of the handheld device 4 towards the patient who is wearing their chosen spectacles. Ideally the patient is seated and encouraged to look directly ahead into the lens 15 of the handheld device 4. To obtain the best result the patient will be encouraged to adopt a natural head position. It is assumed that the patient's head will be held vertically and will not be tilted to one side.

An image of the patient is presented to the user at step 104, in real time, on the LCD display module 22. Aiming guides are superimposed on the image to provide a reference to correctly compose the patient's head in the center of the LCD display module 22.

Housed within the casing 12 is an electro-mechanical tilt sensor (not shown). In an alternative embodiment an accelerometer may take the place of the electro-mechanical tilt sensor. Readings from the tilt sensor are displayed graphically on the LCD display module 22. The user can then adjust the orientation of the handheld device 4 until the readings confirm that the handheld device 4 is being held level. If the orientation of the handheld device 4 is not level, the software will prevent the image from being captured. The user is provided with indicators, for example graphical information displayed on the LCD display module 22 or an audible signal, to confirm that the handheld device 4 is being held at an acceptable orientation.

In addition, the output value from the tilt sensors will be stored when the image is captured. Software running in the handheld device 4 will use the output from the tilt sensors as correction or image scaling factors to adjust the image.

Once the orientation of the handheld device 4 is confirmed as acceptable at step 112, the software activates the image capture key 24b. At which point convergence control is activated at step 114.

When focusing on near objects a patient's eyes rotate inwards. This “convergence” phenomenon can result in a two to three millimetre reduction in the distance between the patient's pupils. In fact, the recommended distance between the patient and the user (between 1.5 and 2.0 metres) will, due to convergence, affect the inter-pupillary distance if the subject focuses on the device itself. Accordingly, the handheld device 4 comprises the convergence control unit 19 for encouraging the patient to focus to infinity, known as an “infinity gaze”, so that convergence does not affect the measurements.

In the first embodiment of the present invention, the convergence control unit 19 comprises a laser which shines a laser beam through a diffuser to create a laser speckle pattern. When the front of the handheld device 4 is viewed by the patient as shown in FIG. 2b, the patient will be looking directly at the convergence control unit 19 and into the laser speckle pattern. At which point the patient's eyes will focus on infinity.

In an alternative embodiment, the patient is encouraged to focus to infinity by the handheld device 4 which, in this embodiment, configured with two light sources positioned at a set distance apart on the front surface of the handheld device 4, facing the subject. The user will instruct the patient to look at the lights and adjust their focus until the two light sources merge into one. This ensures that the eyes are not converged.

A further alternative method of discouraging pupil convergence is to incorporate a reflective surface on the front of the handheld device 4 in which the subject can view their reflected image. This effectively doubles the patient's focal distance and reduces the amount of convergence.

In a further alternative embodiment, it is also be possible for software running on the microprocessor to calculate the amount of convergence based on the patient-handheld device distance measurement and use that value as a correction factor to calculate the distance between the patient's pupils.

When the image capture key is depressed at step 116 the first optical lens assembly 14a and the second optical lens assembly 14b each focuses on the patient and each creates a digital image of the patient which is stored in the memory. The software contains algorithms which are known to the skilled reader, and which process each image as described herein.

An edge enhancement algorithm detects and highlights the edges of each eye in each image at step 118. Then a circle recognition algorithm to detect the iris and/or pupil of each eye in each image at step 120. In addition, at step 122, a dark region algorithm is used to detect the pupil of each eye to confirm the position of the pupils in each image. However, the dark region algorithm may be used instead of the edge enhancement algorithm and/or the circle recognition algorithm to detect the pupils in each image. Examples of the algorithms used in the invention are Kernel based filtering, thresholding and Hough transform algorithms.

Once the irises and/or pupils have been detected using the above algorithms the software runs a least mean square fit algorithm on each detected iris and/or pupil at step 124 to establish the center of each pupil in each image.

In an alternative embodiment of the invention, the handheld device 4 may incorporate a red-eye light source 18, such as a standard camera flash, to encourage the phenomenon of red-eye, a phenomenon wherein light reflects on the retinas which makes the pupils appear red in colour. This phenomenon is used to highlight the patient's pupils, making them easier for the software to identify during image processing. In this embodiment the handheld device 4 captures two images: a “red-eye image”, during the capture of which the red-eye light source 18 flashes, and a standard image which is captured immediately afterwards. The red-eye image is used during the automatic feature recognition process, which filters the image for colours at the red end of the light spectrum. Alternatively, the images mage be “subtracted” to identify the pupils since the pupils will be bright in the red-eye image and dark in the standard image which enables easy pupil identification. The standard image will be used to compile the print output file in order that the images of the patient do not appear with red-eye.

As an alternative embodiment or back-up to the automated steps described above, or a “fine-tuning” mechanism, the user can use the curser keys 24a on the handheld device 4 to move a curser on the LCD display module 22 to indicate the center of each of the patient's pupils. The software has a “zoom” function that allows accuracy up to 1 pixel which equates to approximately 0.1 mm.

As mentioned above, the optimum distance from the patient to the handheld device 4 is between 1.5 and 2.0 metres. In the current embodiment of the invention, using the first optical lens assembly 14a and the second optical lens assembly 14b which are positioned at a known distance apart, the handheld device 4 measures the distance from the handheld device 4 to each of the patient's eyes separately using stereoscopic imaging at step 126. As described above, each optical lens assembly captures an image simultaneously and software on the microprocessor analyses each of the patient's eyes on the two images. The microprocessor analyses and processes the image using standard stereoscopic algorithms to calibrate the system and calculate distances.

Since, in this embodiment, the handheld device 4 calculates the distance from the patient's eyes to the handheld device in a 3D space the software can also make corrections if the patient is facing slightly to the left or right. Software on the handheld device 4 determines a scaling factor by comparing the distance between the patient's eyes on the images against the known distance between the two optical assemblies. Therefore, the handheld device 4 is able to apply measurement units to the approved image and, for example, calculate the patient's pupilary distance. The use of stereo image capture will also allow the possibility of creating 3D images of the subject wearing the spectacle frames, if necessary.

In the second embodiment of the invention, the distance from the patient to the handheld device 4 is measured using ultrasound. The ultrasonic transmitter 16a transmits an ultrasonic signal which is reflected by the patient and received by the ultrasonic receiver 16b. The ultrasonic transmitter 16a and receiver 16b are mounted in the handheld device adjacent to the single optical lens assembly 14 so that the ultrasonic signal can be transmitted towards the patient. Software on the microprocessor then determines the distance from the patient to the handheld device 4.

In an alternative embodiment of the invention, the distance from the handheld device 4 to the patient is determined using a single motor-driven optical lens assembly 14. In this embodiment, the sharpness of the image produced by the lens is assessed by the software on the microprocessor of the handheld device 4. If the image produced is not a sharp image, the software will cause the motor of the optical lens assembly to adjust the optical assembly until a sharp image is achieved. The lens assembly is calibrated so that the software can determine the additional amount by which it has had to drive the lens to achieve a sharp image. This information can then be extrapolated into an accurate distance measurement.

If the distance relationship between the patient and the handheld device 4 falls outside of a predetermined tolerance, the unit 4 will indicate this fact to the user, for example with graphics presented on the LCD display module 22 and/or an audible signal, to instruct the user to move closer or further away from the patient, as appropriate.

In a further alternative embodiment, aiming guides superimposed on the LCD display module 22 can be used approximate the correct distance of the patient from the handheld device 4.

At step 128, the handheld device 4 indicates whether the distance of the patient from the handheld device 4 falls with the parameters mentioned above. If it does not, the user adjusts the distance from the handheld device 4 to the patient until it does so.

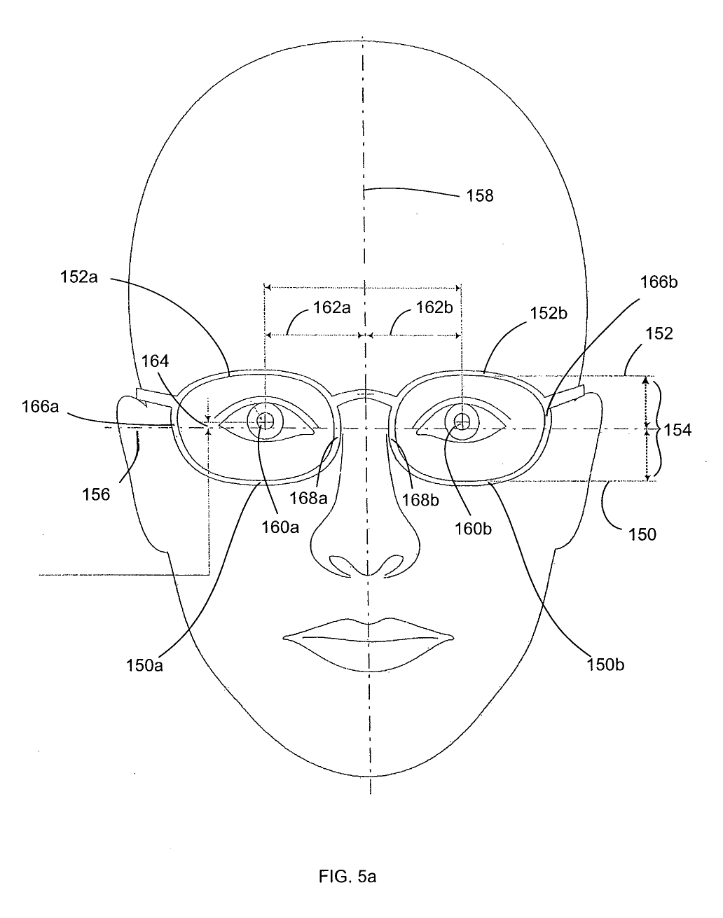

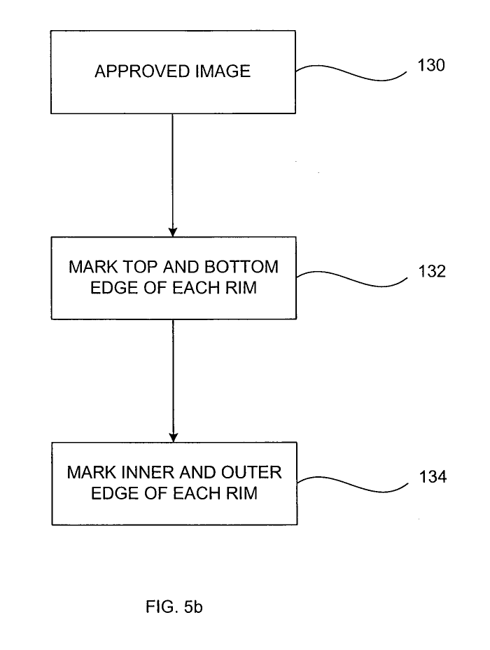

Once the user has completed the above steps an image for approval is presented to the user on the LCD display module 22 and the process of lens selection can begin at step 130, as shown on FIG. 5b. If the image is not correct the process of composing and recapturing the image can be repeated.

In an alternative embodiment, the image capture key 23b has a first and second level of depression. The first level enabling steps 102 to 128 to take place, and a second level where the image is presented to the user on the LCD display module 22, in a similar way to which a digital camera works. The software may be configured in such a way so that it will not be possible to fully depress the key until the correct patient-handheld device distance and level is achieved.

The approved image is presented to the user on the LCD display module 22 at step 130. Superimposed over the approved image are horizontal and vertical cursers which can be moved using the curser keys 24a. Referring to FIG. 5a, the user moves the horizontal curser to the top edge of each frame rim 152a, 152b and bottom edge of each frame rim 150a 150b and marks these positions on the approved image using the select key 24f at step 132. The user does the same to mark the positions of the inner edge of each frame rim 168a, 168b and the outer edge of each frame rim 166a, 166b on the approved image at step 134. The software comprises a “zoom” function that allows accuracy to 1 pixel, equating to approx 0.1 mm.

The distance 154 between the mid-point 152 of the two upper edges 152a, 152b and the mid-point 150 of the two lower edges 152a, 152b is calculated by the software. A horizontal line 156 is positioned midpoint between the lowest point 150 and the highest point 152 and is referred to as the frame datum.

The vertical datum is a notional vertical line 158 positioned on the midpoint of the spectacle frame bridge which is marked on the image using the curser key 24a and the select key 24f.

The centers of the right pupil 160a and the left pupil 160b are detected as described above and, since the handheld device 4 is able to apply measurement units to the approved image, the pupillary distance is known. Therefore, a first distance 162a from the pupil center of the right eye 160a to the vertical datum and a second distance 162b from the pupil center of the left eye 160b to the vertical datum will be calculated by the software.

A vertical distance 164 from the frame datum to the pupil centers of the right pupil 160a and the left pupil 160b. The vertical distance 164 is known as the height above datum (H2).

Pantoscopic tilt is the angle from the vertical plane of the face that the spectacle lenses are positioned. The angle is normally set at between 8 and 10 degrees. Because the image is captured normal to the vertical plane of the face, the pantoscopic tilt angle introduces a foreshortening error to the calculation of vertical dimensions. Therefore, it is necessary to apply a scaling factor to the vertical dimensions measured by the handheld device 4. The software is configured to apply a correction value, assuming a default pantoscopic tilt angle.

The software on the handheld device 4 to superimposes a graphic of concentric circles of pre-determined diameters, which relate to different lenses, registered on the pupil centers of the approved image. This allows the user to select the lens blank size appropriate for the chosen spectacle frames. The software also allows data from the lens manufacturers to be downloaded onto the handled unit 4, allowing non-standard blank sizes to be superimposed over the image.

The data communications port 26, or in another embodiment the docking station 6, enables the handheld device 4 to be connected to a PC or other device capable of connecting to the Internet. Connection to lens manufactures' web portals enables downloading lens manufacturer geometric data of the various lens types available. Accordingly, the user has the option of choosing a number of different lens types from a variety of lens manufacturers. The type of lens, material it is manufactured from and the patient's prescription will all result in different lens thicknesses. By knowing the eventual thickness, the optician is able to make a judgement on the appropriateness of a particular lens type for the spectacle frames chosen. Choosing the wrong lens will result in overly thick, unsightly lens edges. The software is able to calculate the eventual lens edge thickness for the chosen frame profile using utilising SAG formulas which are well known methods of determining curves using different refractive indices of lenses and taking into account the distance from the optical center of the lens. When calculating the thickness at different points of the lens, a circular cursor appears on the LCD display module 22 which can zoom in and out of the approved image to display the corresponding edge thickness. If deemed too thick, the user has the ability to choose a different lens type.

The communications features of the invention mentioned above allows the downloading or ordering of lenses directly from the lens manufacturers, via the handheld device 4. To achieve this, the handheld device 4 incorporates a means of interfacing with manufacturer on-line ordering web portals. In a further embodiment, this may be achieved by incorporating a modem in the device, which can connect to the Internet directly or via a wireless connection.

Once the user and patient are satisfied with the lens and frame selection, the user selects a function wherein the software converts the approved image into an output file which is in a format suitable for printing on the print output device 8 when the unit 4 is docked in the docking cradle 6. The image recognition software, running on the microprocessor, automatically corrects the image for level and scale and applies automatic contrast and brightness filters. In an alternative embodiment, communication between the handheld device and the print output device 8 is via a wireless connection. Alternatively, the docking cradle 6 includes a “direct print” key, which will allow printing to the print output device 8 with a single key press when the handheld device 4 is docked in the decking device 6. Alternatively, the docking cradle 6 is integrated with the print output device 8.

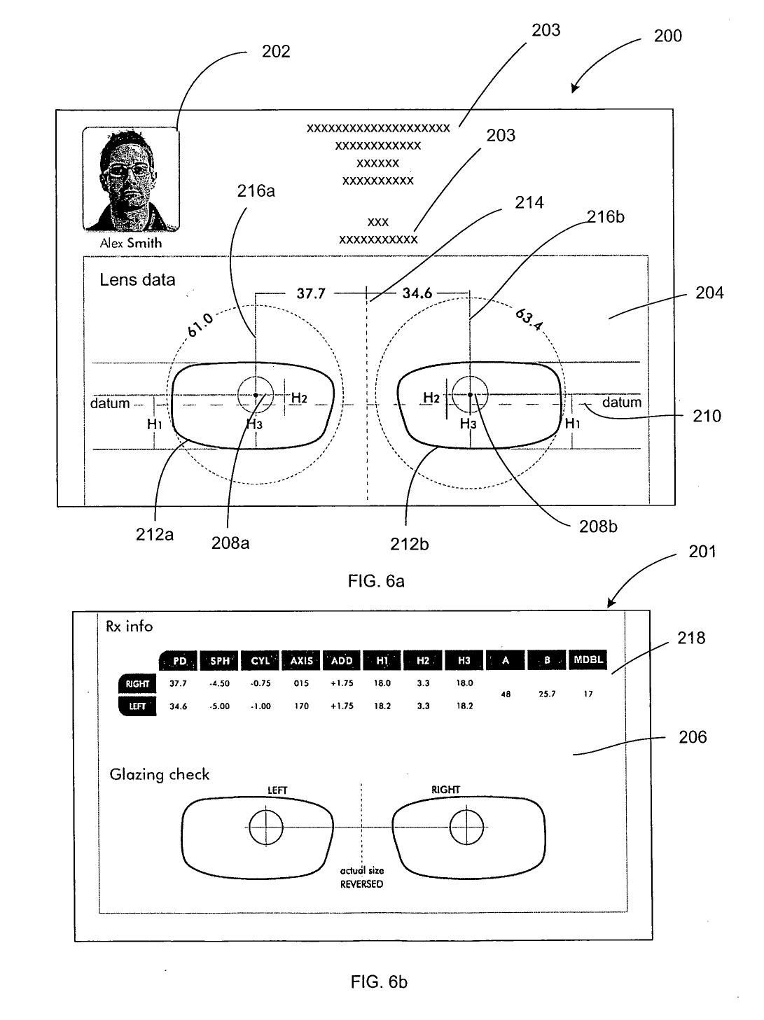

The output file, which is in a customised format and will work only with the print output device 8, comprises a first printed file 200, shown in FIG. 6a, which contains a small-scale inset head shot 202 of the patient wearing the frames; a customisable area where the optician retailer may enter its contact details, for example; and a cropped 1:1-scale image of the eye area 204 (mid-forehead to tip of nose). A reversed image 206 for checking the frames once they have been glazed is shown in FIG. 6b.

The first printed file 200 contains graphics superimposed on the images above showing the pupil centers 208a, 208b; the horizontal frame datum 210; the lens edge/frame profile 212a, 212b; the vertical datum 214; vertical lines through the center of the pupils 216a, 216b.

The reverse of the printout 201 of FIG. 6a, shown in FIG. 6b, also displays the patient's spectacle prescription 218. PD is the distance from each of the patient's eyes to the vertical datum 214; SPH, CYL, AXIS and ADD are the well known spectacle prescription abbreviations for sphere, cylinder, axis and additional refractive power; H1 is the distance from the pupil center to the user-selected lower frame edge; H2 is the height above datum; H3 is vertical distance from the pupil center to the lower lens edge as detected automatically in a further embodiment of the invention; A is the horizontal length of each lens; B is the vertical distance of each lens; and MDBL is the minimum distance between the lenses.

It will be apparent to the skilled user that the various embodiments of the present invention may be readily combined. The present invention may be embodied in other specific forms without departing from its essential attributes. Accordingly, reference should be made to the appended claims and other general statements herein rather than to the foregoing specific description as indicating the scope of the invention.

1. A device for aligning a lens with the eye of a patient, the device comprising:
image capture apparatus for capturing and storing an image of a patient wearing spectacles; and
a processor for determining on the image the center of a pupil of the patient, and indicating on a display the position of the lens over the eye of the patient wherein the optical center of the lens is aligned with the pupil of the patient.

2. The device of claim 1, wherein the processor comprises an edge enhancement algorithm for highlighting edges in the image.

3. The device of claim 1, wherein the processor comprises a circle recognition algorithm for detecting circular shapes in the image.

4. The device of claim 1, wherein the processor comprises a dark recognition algorithm for detecting dark areas in the image.

5. The device of claim 1, wherein the processor comprises an algorithm for detecting the center of the pupil of the patient in the image.

6. The device of claim 1, wherein the processor comprises a red-eye light source.

7. The device of claim 6, wherein the red-eye light source is a standard camera flash.

8. The device of claim 6, wherein the device comprises comparison apparatus for comparing a standard image and a “red-eye” image.

9. The device of claim 1, wherein the device comprises a range-finder for calculating the distance from the device to the patient.

10. The device of claim 9, wherein the range-finder comprises a double optical assembly which are positioned a known distance apart, and an optical processor for calculating the distance from the device to the patient using stereoscopic imaging.

11. The device of claim 9, wherein the range-finder comprises:
a single optical assembly wherein the assembly is motor-driven; and
a focus detector which is arranged to drive the assembly to achieve a sharp image;
wherein the single optical assembly is calibrated with the focus detector so that the distance from the device to the patient is calculated.

12. The device of claim 9, wherein the range-finder comprises an ultrasonic transmitter for transmitting an ultrasonic signal and an ultrasonic receiver for receiving the ultra sonic signal, and an ultrasonic processor for calculating the distance from the device to the patient.

13. The device of claim 9, wherein the range-finder comprises aiming guides superimposed on the display.

14. The device of claim 1, wherein the device comprises a pointer for indicating, on the image, edges of a spectacle frame for the lens.

15. The device of claim 1, wherein the device comprises a convergence control unit.

16. The device of claim 15, wherein the convergence control unit comprises a laser speckle generator.

17. The device of claim 15, wherein the convergence control unit comprises a first light source and a second light source.

18. The device of claim 15, wherein the convergence control unit comprises a reflective surface.

19. The device of claim 15, wherein the convergence control unit comprises a convergence processor for correcting convergence.

20. The device of claim 1, wherein the device comprises an orientation detector for detecting the orientation of the device or scaling the image.

21. The device of claim 20, wherein the orientation detector is an electromagnetic tilt sensor.

22. The device of claim 20, wherein the orientation detector is an accelerometer.

23. The device of claim 1, wherein the device comprises cursor keys and a select key for moving a cursor on the display of the device for indicating edges of the spectacle frames.

24. The device of claim 1, wherein the device is arranged to calculate dimensions on the image.

25. The device of claim 24, wherein the dimensions are one or more of the frame datum, vertical datum, PD, H1, H2, H3 and MDBL.

26. The device of claim 1, wherein the device is arranged to calculate pantoscopic tilt.

27. The device of claim 1, wherein the device is arranged to superimpose a circle, the image representing a lens, over the eye of the patient on the image.

28. A system comprising:
a device according to claims 1;
a docking station engageable the device arranged to communicate with and provide power to the device; and
a print output device.

29. The system according to claim 28, wherein the print output device is arranged to print an output file corresponding to the image on the display.

30. A method for aligning a lens with the eye of a patient, the method comprising:
capturing and storing an image of a patient wearing spectacles on a device;
processing the image to determine the center of the pupil of the patient; and indicating the correct position of the optical center of the lens over the pupil of the patient on a display of the device.

31. The method of claim 30, wherein the method comprises inducing an infinity gaze in the patient.

32. The method of claim 30, wherein the method comprises a user of the device altering the position of the lens in the image.

33. The method of claim 30, wherein the method comprises measuring the distance from the device to the patient.

34. The method of claim 33, wherein the method comprises scaling the image using the distance from the device to the patient.

35. The method of claim 30, wherein the method comprises indicating edges of the spectacles on the image.

36. The method of claim 30, wherein the method comprises selecting a lens blank from a selection of lens blanks illustrated on the display.

37. The method of claim 36, wherein the method comprises connecting to the Internet to download the selection of lens blanks.

38. The method of claim 30, wherein the method comprises calculating dimensions on the image.

39. The method of claim 38, wherein the dimensions are one or more of the frame datum, vertical datum, PD, H1, H2, H3 and MDBL.

40. The method of claim 30, wherein the method comprises communicating the type of lens and its position relative to the spectacles to a manufacturer.

41. A method for aligning a spectacle lens with the eye of a patient, the method comprising:
calculating the distance between the patient and a device using a double optical assembly;
capturing and storing an image of the patient wearing spectacles on a device;
processing the image to determine the center of the pupil of the patient; and indicating the correct position of the optical center of the lens over the pupil of the patient on a display of the device.

42. A device for aligning a spectacle lens with the eye of a patient, the device comprising:
a double optical assembly for calculating the distance from the device to the patient;
image capture apparatus for capturing and storing an image of a patient wearing spectacles; and
a processor for determining on the image the center of a pupil of the patient, and indicating on a display the position of the lens over the eye of the patient wherein the optical center of the lens is aligned with the pupil of the patient.
